(19) United States
(12) Patent Application Publication
(10) Pub. No.: US 2024/0008721 A1
(43) Pub. Date: Jan. 11, 2024

(54) BENDING SECTION FOR AN ENDOSCOPE

(71) Applicant: AMBU A/S, Balleup (DK)

(72) Inventor: Morten SØRENSEN, Ballerup (DK)

(73) Assignee: AMBU A/S, Ballerup (DK)

(21) Appl. No.: 18/219,469

(22) Filed: Jul. 7, 2023

(30) Foreign Application Priority Data
Jul. 8, 2022 (EP) 22183911.1

(51) Int. Cl. A61B 1/005

(52) U.S. Cl. CPC A61B 1/0055; A61B 1/0057

(57) ABSTRACT

An endoscope including a handle and an insertion cord extending from the handle, the insertion cord including a main tube, the tip part, and a bending section body. The bending section body includes segments interconnected by hinges. The segments include a proximal end segment, a distal end segment and intermediate segments. A steering wire passes through the proximal end segment and at least some of the intermediate segments, forming an active portion of the bending section, and does not pass through segments distal of the active portion.

### CROSS-REFERENCE TO RELATED APPLICATIONS

This application claims priority from and the benefit of European Patent Application No. EP 22183911.1, filed Jul. 8, 2022, which is incorporated by reference herein in its entirety.

### TECHNICAL FIELD

The present disclosure relates to endoscopes, in particular but not exclusively to disposable EndoBronchial UltraSound endoscopes, normally abbreviated with the acronym EBUS endoscopes.

### BACKGROUND

Like many other endoscopes, EBUS endoscopes comprise a proximal handle from which an insertion cord extends. The insertion cord normally comprises a bendable main tube to the distal end of which a highly flexible bending section is connected. The bending section normally comprises a number of segments connected by hinge members so as to allow it to be articulated, that is to say bent out of a straight as-made configuration. In the straight as-made configuration, the longitudinal axis of the bending section is normally aligned with the longitudinal axis of the main tube. The distal end of the bending section, in turn, is connected to a tip member with features providing desired functionalities such as illumination, vision, exit ports for tools, liquid instillation, fluid insufflation, suction etc. Commonly owned International Application No. PCT/DK2021/050122 (WO2021/219178) and U.S. Pat. No. 11,471,031, issued Oct. 18, 2022, both incorporated by reference herein in their entirety, describe bending sections connected by live hinges.

In EBUS endoscopes one of the features is an ultrasound transceiver allowing to look into the tissue behind e.g. the bronchial wall. Furthermore, in EBUS endoscopes, the tip member may comprise attachment means, such as recesses, for the attachment of a balloon surrounding the ultrasound transceiver. The balloon may then be filed with liquid from a port in the tip part so as to provide a good match of acoustic impedance to the surrounding tissue that is to be ultrasonically probed. The proximal handle is adapted to be gripped by a hand of an operator. The handle may comprise an operating member allowing control of the bending section, so that the insertion cord may be maneuvered during insertion into a patient e.g. into the bronchia.

As e.g. known from U.S. Pat. No. 6,409,666 the ultrasound transceiver in the tip member emits and receives in a sideways direction i.e. at high angles transversely to the overall longitudinal direction of the endoscope, in the following understood as the center axis of the main tube when in a straight as made configuration. That is to say, not bent by external forces. This allows the ultrasound transceiver to look through the bronchial wall and into the tissue behind, e.g. for locating lymph nodes. The vision camera on the other hand is more forward looking, allowing the operator to maneuver the tip of the endoscope to a desired position in the bronchia. The working channel at the exit port in the tip is also placed at an angle in order to allow a tool, in particular a biopsy needle, to exit at an angle allowing penetration of the bronchial wall into lymph nodes behind the bronchial wall for sampling while at the same time being visible by the ultrasound transceiver.

One problem in this respect is to press the emission face of the ultrasound transceiver into proper engagement with the bronchial wall, inter alia to avoid air filled gaps between the emission face and the bronchial wall that will cause sound reflections because of poor match of acoustic impedance between the various media, e.g. air vs. waterfilled tissue. Although this may partially be remedied by using a liquid fillable balloon around the tip a good engagement force is still necessary.

Another problem is that the surface of the ultrasound transceiver is curved and different parts along the length of the emission face will engage the tissue depending on the dimensions of the surrounding bronchial passage.

Furthermore, since the bronchia branches into narrower and narrower passages, it may often be difficult for more proximal parts of the bending section or the main tube to properly engage a bronchial wall. Without a more proximal part also abutting the wall it is not possible to provide any proper engaging force with the tip. This may also influence the force and which place on the surface engages the ultrasound transducer.

As known from U.S. Pat. No. 9,332,961, one way to overcome this has to been to provide the part of the tip that accommodates the ultrasound transducer with a predetermined angle, i.e. that the back of the tip opposite the front face of the curved ultrasound transceiver has an angle with respect to the center axis. This, however, does not solve all of the above problems satisfactorily. Inter alia the cross-wise dimensions of the distal end of the endoscope increase because of the tip part being bent out of the plane.

### SUMMARY

It is an object of the present disclosure to provide an endoscope which shall reduce or avoid the disadvantages of the related art. More specifically, the problem addressed by the present disclosure is how navigate the endoscope to a target site and press the emission face of the ultrasound transceiver into proper engagement with the target site to increase the quality of the ultrasound image.

According to a first aspect of the present disclosure, this object is achieved by an endoscope comprising a bending section body having hinges and a steering wire attached to a segment of the bending section body proximally of at least a distal-most hinge.

By attaching the pull-wires to an intermediate segment only, rather than to the distal end segment, the hinge towards the distal end segment may bend independently of the bending of the remainder of the bending section. Accordingly, when the bending section bends the intermediate segments in a direction towards a target site, the tissue between the target site and the tip part may press the distal end segment, to which the tip part is attached, into the opposite direction, thus ensuring a better, i.e. more parallel alignment between the tissue and the tip part. That is to say, better alignment results in better signal quality by the ultrasound transceiver with respect to a specific direction towards the target site, if the tip part comprises an ultrasound transceiver.

The endoscope may also have a proximal handle, an insertion cord extending from said handle towards a distal end of the endoscope, the insertion cord comprising a main tube, a tip part, and a bending section body arranged between said main tube and said tip part. The bending section body comprises segments interconnected by hinge members arranged in a hinge plane, said segments comprising a proximal end segment, a distal end segment and intermediate segments. The proximal end segment and at least some of the intermediate segments comprise pull-wire passages. The steering wire(s) (or pull-wire(s)) are attached and secured to one of the intermediate segments.

According to an embodiment of the first aspect of the disclosure, the tip part comprises an ultrasound transceiver. Though the alignment and engagement with the bronchial wall may have other uses, it is especially advantageous to ensure good contact with no or few acoustic reflections between the ultrasound transducer and the tissue of and behind the bronchial wall. This in turn yields good ultrasound images.

According to an embodiment of the first aspect of the disclosure, the steering wire passages are arranged in pairs in a plane perpendicular to said hinge plane. This allows efficient conversion of the pull into torque about the hinges.

According to an embodiment of the first aspect of the disclosure, the bending section body comprises a first set of segments, through which the pull-wires pass, and a second set of segments, through which the pull-wires do not pass. The second set of segments are positioned distally of the first set of segments. The segments in the first set of segments may be referred to as “actively controlled” segments, referencing active control via the steering wire(s). The segments in the second set of segments may be referred to as “passive” segments, because they are not actively, or directly, steered. The pull-wire(s) is/are connected to the most distal segment of the first set of segments. This allows even better alignment of the tip part by allowing the tip part to self-orient relative to the target site. In one variation, the number of segments in said second set of segments is selected from the group comprising 1, 2, 3, 4, 5 or 6. This has been found to be sufficient when the tip part comprises said ultrasound transceiver. In one example, the second set of segments comprises two segments, thereby providing two sets of passive hinges. In another example, the second set of segments comprises three segments, thereby providing three sets of passive hinges. The second set of segments may also comprise just one segment, the distal end segment, providing one set of passive hinges. A set of hinges comprises all the hinges that connect two segments. In a single-plane endoscope, where the bending section bends in two directions along the plane, two adjacent segments are generally connected by two or more hinges.

According to a variation of the foregoing embodiments of the first aspect of the disclosure, the bending section body is provided as an integrally molded item and the hinges are provided as part of the integrally molded item, and thus may be referred to as “live” hinges. This takes advantage of the fact that such integrally molded hinges are elastic with a certain resiliency allowing them to transfer a force from the actively controlled bending section segments to the passive bending section segments. This in turn ensures good engagement between the tip part and the target site.

According to a variation of the foregoing embodiments of the first aspect of the disclosure, the hinges distal of the steering wire are wider (circumferentially) than at least some of the hinges between actively controlled segments. This allows the hinge to be more resistant to bending and hence increases the force with which the tip with the ultrasound transceiver may be pressed against the target site.

According to a second aspect of the disclosure, the object is achieved by a visualization system comprising a display unit and an endoscope according to any one of the endoscope embodiments disclosed herein, including all variations and examples thereof.

By using the endoscope according to the first aspect in such a system, better visual and/or ultrasonic targeting of objects of interest behind a bronchial wall may be achieved.

According to an embodiment of the second aspect of the disclosure, the display unit comprises an integrated display. Having an integrated display gives lesser items to handle in the set-up for patient examination.

According to an embodiment of the second aspect of the disclosure, the system further comprises an ultrasound control box for sending signals to an ultrasound transceiver and receiving signals from an ultrasound transceiver. This provides interchangeability of not only the ultrasound control box but also of the display device and other parts of the system, and they may not depend on each other.

According to an embodiment of the second aspect of the disclosure, the ultrasound control box comprises image processing electronics. This allows the ultrasound box to be a self-contained unit with or without integrated display.

### BRIEF DESCRIPTION OF THE DRAWINGS

The following figures illustrate embodiments of the disclosure. The disclosure is not limited to the embodiments described below. Other embodiments, combinations of embodiments and modifications thereof may be provided within the scope of protection defined by the claims.

FIG. 1 shows an embodiment of a system, according to the disclosure, comprising an EBUS endoscope, a display unit and an ultrasound box;

FIG. 2 shows an exploded side view of the main tube, bending section and a tip part of the EBUS endoscope of FIG. 1;

FIG. 3 shows an embodiment of a steering wire for operating the bending section of FIG. 2;

FIG. 4 is a perspective view from the distal end of a bending section body of the bending section of FIG. 2;

FIG. 5 is a perspective view of the bending section body of FIG. 4 from a slightly different angle and in partial section;

FIG. 6 shows a cross-section of the bending section of FIG. 2;

FIG. 7 shows the distal end segment as in FIG. 4 but also showing a steering wire;

FIG. 8 shows a passive hinge;

FIG. 9 is a schematic diagram of stiffening pins and a circumferential wall showing passages for the stiffening pins;

FIG. 10 is a schematic diagram of stiffening pins and a transverse wall showing passages for the stiffening pins;

FIG. 11 is a schematic side view of a stiffening pin positioned through the transverse wall of FIG. 10, of a distal end segment, and bend limiting shoulders;

FIG. 12 shows a longitudinal cross-section of the bending section of FIG. 2 and also a longitudinal cross-section of a bending section sleeve;

FIGS. 13 and 14 show cross-sections of a stiffening bending section sleeve;

FIG. 15 shows a cross-sections of a passive portion of a bending section body;

FIG. 16 shows an enlarged side view of the tip part of the tip part of FIG. 2; and

FIG. 17 schematically shows how the segments of the bending section would bend within a duct or passage, such as in the bronchia.

### DETAILED DESCRIPTION

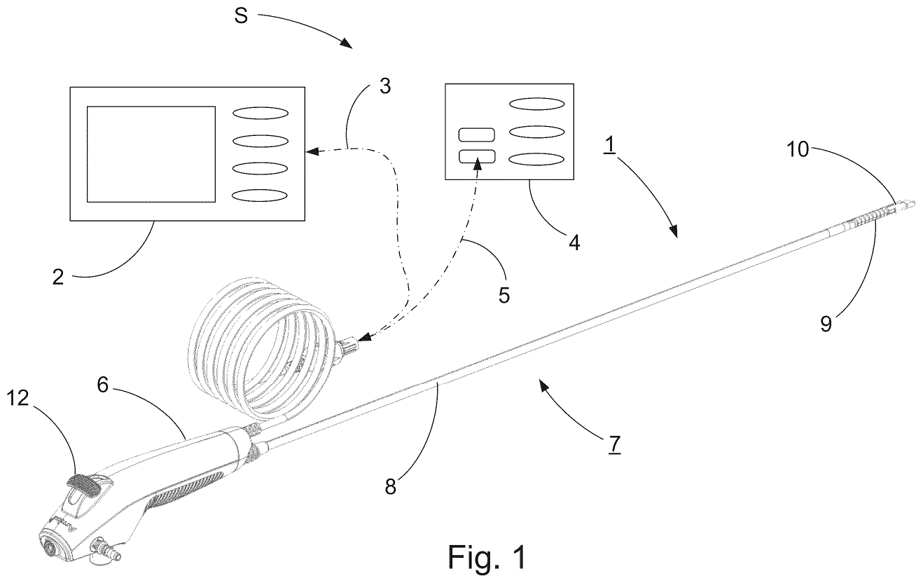

Turning first to FIG. 1, a visualization system S including an EBUS endoscope 1 according to the disclosure and a display device 2, to which the endoscope 1 is connectable as indicated with the dashed double arrow 3, is provided. The connection to the display device 2 need not be direct but may be via a display unit with image processing capabilities to which the display unit 2 is connected. When, as envisaged, the endoscope is an EBUS endoscope the system further comprises an ultrasound control box 4 to which the endoscope is also connectable, as indicated by the double arrow 5. The ultrasound control box 4 comprises signal processors and other electronics for sending signals to the ultrasound transceiver and receiving signals therefrom. The electronics may also include processors as CPUs or FPGAs for processing the ultrasound images to be displayed. The ultrasound image could be sent to a separate display, or be sent to the image display, e.g. as picture-in-picture, if the display device has only a single screen, or the ultrasound control box may have its own integrated display. Additional details regarding the display device 2 are provided further below.

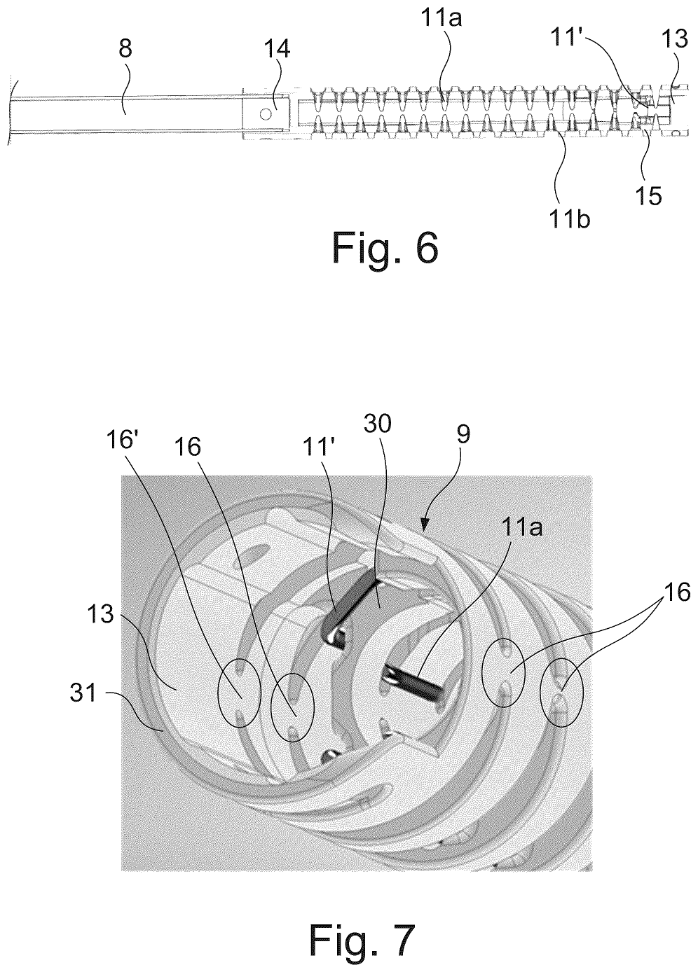

The endoscope 1 comprises a proximal handle 6 from which an insertion cord 7 extends towards to the distal end of the endoscope 1. The insertion cord 7 comprises several parts, such as a main tube 8, a bending section comprising a bending section body 9, and a tip part 10. Pull-wires, or steering wires, 11 (shown in FIG. 3) for actively bending the bending section body 9, are attached to the bending section body 9 and run from the bending section body 9 through the main tube 8 to an operating member 12, where they are also secured, thus allowing the bending section body 9 to be bent in different directions by tensioning or slacking the pull-wires 11 in a per se well known manner. This not only allows the distal end of the endoscope to be maneuvered to a target site but also to force the ultrasound transceiver 19, seen in FIG. 6, of the tip part 10 into engagement with a tissue, such as a bronchial wall, at the target site. The bending section body 9 is an open construction and is therefore normally covered by a sheath which, however, has been entirely omitted in the figures so as not to disturb the illustration of the bending section.

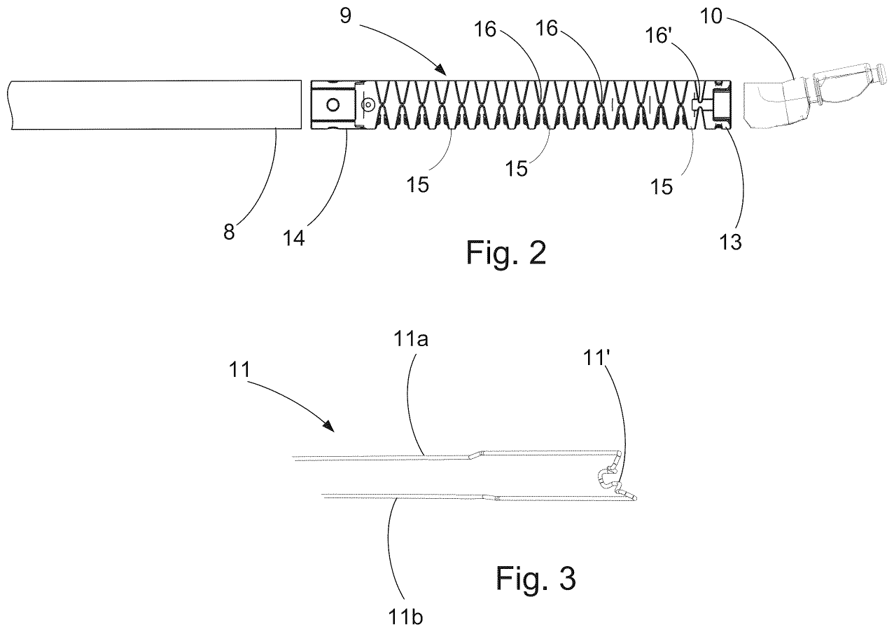

FIG. 2 is an exploded view of the insertion cord 7, showing the tip part 10, the bending section body 9, and a distal part of the main tube 8. The bending section body 9 includes a distal end segment 13, a proximal end segment 14, and intermediate segments 15 between the distal end segment 13 and the proximal end segment 14. The segments are connected by short bridges of material serving as live hinges 16, 16′. Once assembled, the tip part 10 is attached to the distal end segment 13 and the main tube 8 is attached to the proximal end segment 14. The bending section 9 is preferably integrally molded as a single piece member of polymer material. The elastic properties of the polymer material allow the short bridges to exhibit some resiliency so that the hinges 16, 16′ bias the bending section towards a straight configuration, as shown in FIGS. 1, 2, and 6.

FIG. 3. shows an embodiment of a pull-wire or steering wire 11. The pull-wire 11 as shown is constituted by a single wire having sections 11a and 11b connected by an interconnection portion 11′ that is wrapped though a series of bends to secure firm attachment to the distal-most actively controlled segment. The interconnection portion 11′ may also be secured with an adhesive. This can also be seen in the cross-sectional view of FIG. 7. In an alternative embodiment the interconnection portion 11′ is removed and distal ends of the wire sections 11a and 11b are secured to the distal-most actively controlled segment by any known means, such as epoxy potting and/or bending the distal ends thereof, as is known in the related art. Since the wire sections 11a and 11b are attached to the intermediate segment 15, the wire sections 11a and 11b function as independent wires. By pulling at the proximal ends thereof it cannot be determined whether there is two individually secured wires or two free ends of a single wire. So, for practical and kinematic purposes, the wire sections 11a and 11b may also be referred to as pull-wires 11.

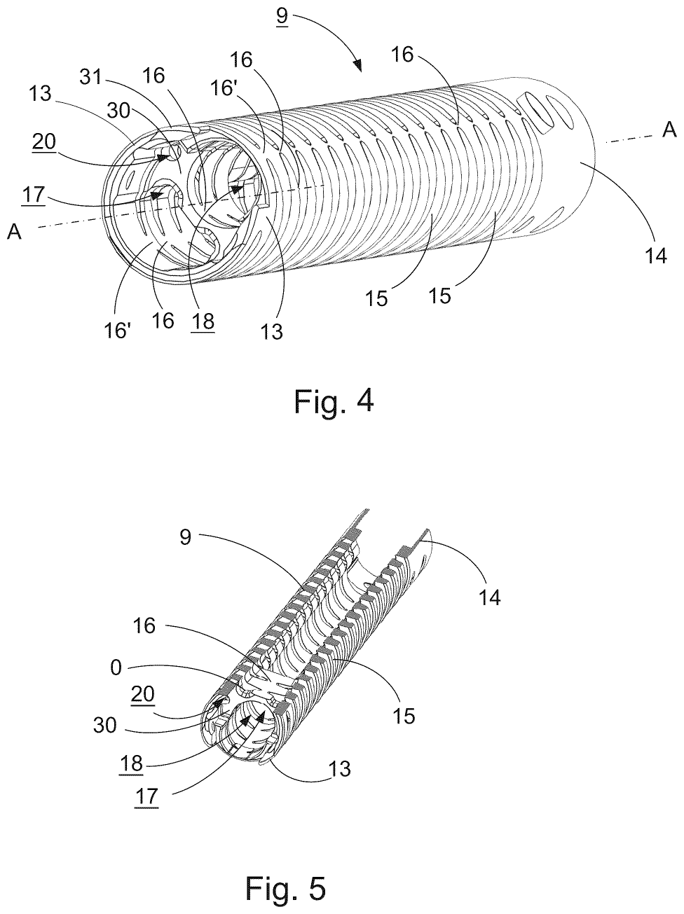

FIG. 4 is a perspective view of the bending section body 9. FIG. 5 is a side view of the bending section body 9 sectioned across a passage 17. FIG. 6 is a perspective view of the bending section body 9 assembled with the steering wires 11 and the main tube 8. In the present embodiment an intermediate segment comprises a wall 30 that is transverse to a longitudinal, central, axis A-A of the bending section body 9. The wall 30 is connected to and extends from a circumferential wall 31. Cut-outs along the circumferential wall 31 define the live hinges 16. The wall 30 comprises two passages 17, 18 arranged symmetrically about the hinge plane so that the hinges between adjacent segments, or hinge set, comprise three bridges, two circumferential bridges and one bridge, denoted by numeral 16″ (e.g. intermediate hinge 16″), coincident with the extension of the wall portion between the passages 17, 18. The passage 18 is preferably circular in order to accommodate a working channel tube (not shown) whereas the passage 17 is preferably somewhat kidney shaped and suitable for accommodating the quite substantial number of electrical wires for the array of transducers in the ultrasound transceiver 19, as well as optical fibers or electrical wires for illumination and electronic imaging of the visual part of the endoscope 1. The wall 30, furthermore, comprises a pair of, smaller, steering wire passages 20 (only one visible in FIG. 4) for the pull-wire 11 used to bend the bending section body 9, and a pair of cut-outs 20′ (shown in FIG. 5). The steering wire passages 20 and the pull-wire 11 are arranged in a plane crosswise to the plane in which the hinges 16 lie. To secure the steering wire 11, the portion 11a is woven through one of the cut-outs 20′ proximally, behind the wall 30, and then distally through the second cut-out 20′, so that the intermediate portion 11′ is proximal of the wall 30 of the distal-most actively-controlled segment. The wire sections 11a and 11b (or pull-wires 11) are then threaded proximally through the steering wire passages 20. As shown in FIG. 4, the live hinge 16′ extends a longer distance along its width, circumferentially, than the live hinge 16 next to it. The wall 30, as shown, is located on the distal-most intermediate segment 15 rather than in the distal end segment 13.

Thusly, the pull-wire sections 11a, 11b are not attached to the distal end segment 13. Pulling the pull-wires 11 will thus not force any bending of the hinge 16′. The hinge 16′ will only bend if an external force in a crosswise direction of the hinge plane is applied to the distal end segment 13. This will happen if the hinge segment 13 itself or the tip part 10 which is rigidly attached to the end segment 13, is forced into engagement with an external object, such as a bronchial wall 22, as can be seen schematically in FIG. 17.

Although the steering wire passages 20 are shown as holes in the wall 31, the steering wire passages 20 can also be cut-outs with an open side adjacent the passages 17 or 18. The manner in which the steering wire 11 passes through the bending section body 9 is irrelevant to the invention.

As shown in FIG. 5, the cross-section is taken along the passage 17. This passage 17 does not run as a separate passage along all the intermediate segments, but rather joins the passage 18 to form a single passage accommodating both. This joining of the passages would also be possible in the previously described embodiment of FIG. 3. In other words, a most distal wall 30 is provided to secure the pull-wires and proximally of the distal-most wall 30 additional walls may be provided. On the other hand, reducing the number of walls may reduce inherent friction and does increase the flexibility of the bending section or reduce the force required to bend it.

In a variation of the present embodiment, the wall 30 is positioned in a different intermediate segment, such that a distal intermediate segment may also be passive, and allowing for two sets of hinges to be distal of the steering wire 11.

In another variation or example of the present embodiment, the portion of the wall 30 intermediate the passages 17 and 18 is removed, thus the peripheries of the two passages are connected.

In another variation or example of the present embodiment, the intermediate hinges 16″ are omitted, at least in some segments, and only circumferential hinges 16′ are provided in those segments.

In another variation or example of the present embodiment, the distal ends of the wire sections 11a and 11b are bonded to the wall 30, without the intermediate portion 11′.

To yield a good abutment force of the ultrasound transceiver against the bronchial wall 22 the hinge 16′ connecting the distal end segment 13 to the intermediate segments is preferably more resilient/stiffer than the hinges 16 between the intermediate segments 15. This may be achieved by the hinge 16′ being made longer in the circumferential direction of the bending section body 9 than the hinges 16. Additional ways to make the hinges 16′ more resilient/stiffer are discussed below.

In this respect it should be noted that the pull-wires 11 do not need to be attached to the most distal intermediate segment 15. Rather it could be attached to a more proximal intermediate segment 15 so that not only the distal end segment but also one or more of the intermediate segments 15 will be able to bend under external force rather than under the direct influence of the pull-wires 11. In other words, the bending section would comprise a set of segments 14, 15 forming an active bending section part and a set of segments 13, 15 forming a passive bending section part. Accordingly, the intermediate segments 15 would comprise a proximal set of intermediate segments 15 forming part of the active bending section, and a distal set of intermediate segments 15 forming part of the passive bending section. Depending on the specific application for the endoscope 1, the passive bending section could then include just the most distal intermediate segment 15 or up to e.g. five intermediate segments 15 towards the distal end segment 13. The hinges 16′ between these intermediate segments 15 could also have increased resiliency, e.g. by making them the same length at the hinge 16′ between the most distal intermediate segment 15 and the distal end segment 13.

As indicated above, the bending section body comprises live hinges and a steering wire attached to a segment of the bending section body proximally of at least a distal-most hinge. An example is shown in FIGS. 6 and 7, where the steering wire is attached to the most distal intermediate segment. A circumferential wall 31 is also identified. The thickness of the wall can also be the thickness of live hinges. The distal-most hinges are the hinges connecting the distal-most intermediate segment to the distal end segment, therefore at least the distal end segment is a passive segment and the hinges connecting it to the intermediate segment immediately proximal of it are deemed passive hinges. Stated differently, the steering wire is attached to one of the intermediate segments, and the steering wire does not extend through segments distal of the one of the intermediate segments to which the steering wire is attached and also not parallel to hinges distal of the one of the intermediate segments to which the steering wire is attached. Thus, for example, if the steering wire is attached to seventh of eight intermediate segments, the eighth intermediate segment and the distal end segment are passive segments, and the hinges attaching them to each other and to the seventh intermediate segment are passive hinges. The other hinges are active hinges. The remaining segments are actively controlled with the steering wire and comprise the first set of segments, through which the steering wire or wires pass, and are interconnected by the active hinges.

As mentioned, the bending section comprises the bending section body and a bending section sleeve positioned over the bending section body. The bending section comprises an active portion and a passive portion distal of the active portion. The active portion comprises a first set of segments, through which the steering wire passes, and at least one segment through which the steering wire does not pass. The steering wire is connected to a most distal segment of the first set of segments. The at least one segment through which the steering wire does not pass is in the passive portion. All the segments distal of the most distal segment of the first set of segments form the passive portion.

In some variations of the foregoing embodiment, the passive portion of the bending section comprises at least one stiffening feature configured to increase resistance to bending relative to the bending section without the stiffening feature.

In one example of the present variation, the hinges are live hinges and the stiffening feature comprises live hinges that are stiffer than live hinges in the active portion.

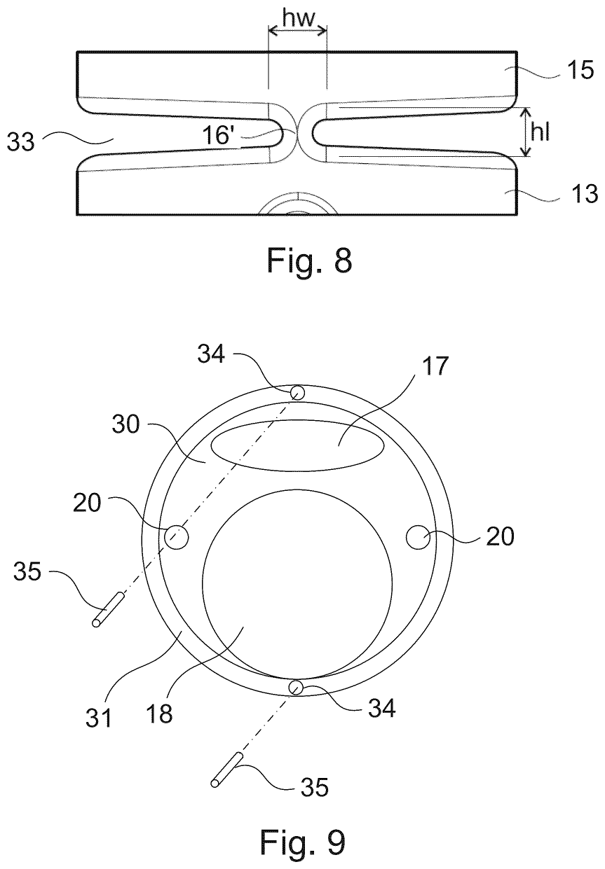

The passive hinges may be more resistant to bending than the active hinges, and the increased resistance to bending may be achieved in a variety of ways. As indicated above, the passive hinges may be circumferentially wider than at least some of the active hinges, as shown in FIG. 4 for example. This allows the passive hinges to be more resistant to bending. FIG. 8 shows a live hinge 16′ to illustrate the width hw and the length hl. Decreasing the length, along the central axis of the bending section body 9, and increasing the thickness, will also increase resistance to bending. The width and length can be modified by changing the size of the cut-outs in the peripheral wall 31, which is accomplished by modifying the mold in which the bending section body is molded. The mold comprises two parts that form a cavity and cores inserted in the cavity to form the steering wire passages and the other passages. Increasing the thickness of the passive hinges can be accomplished, for example, by modifying the mold cavity to increase the cross-section of the cavity where the passive hinges are formed, so that more material is molded between the segments, so that the thickness of the passive hinge is greater than the thickness of the circumferential wall 31. A cut-out 33 is shown, which separates two segments and defines, together with an opposite cut-out, the live hinge 16′. Circumferential wall surfaces on opposite sides of the cut-out 33 may touch if the bending angle of the live hinge is large enough.

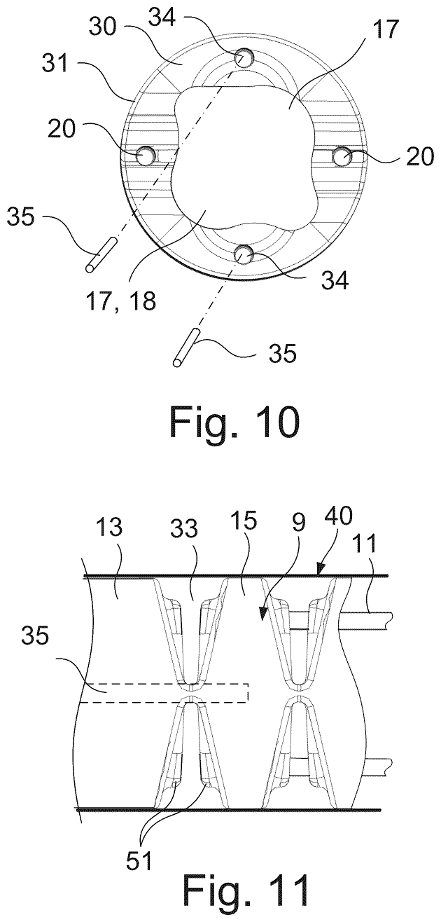

The resistance to bending can also be increased by pinning the passive hinges. FIGS. 9 to 11 show pinning examples. FIG. 9 shows a schematic diagram of a cross-section of the bending section body 9 showing passages 34 adapted to receive stiffening pins 35. Pinning can be achieved in a variety of ways and comprises combining a hinge with the stiffening pin. The hinge can be formed like the active hinges. The stiffening pin can be metal or a rigid polymer. In one example, passages 34 are provided through the hinges, as shown in FIG. 9, so that stiffening pins 35 extend into the circumferential wall 31 and through the hinge. The passages are made by inserting additional cores/rods into the mold cavity. A variation of the above allows for bulging the thickness of the circumferential wall and hinge to provide volume for the stiffening pin passages. In another example, as shown in FIGS. 10 and 11, the transverse wall 30 is provided in the passive segments, similar as the transverse wall 30 of the active segments, and is provided with the passages 34, which on the hinge plane. The stiffening pins 35 are then inserted and secured in the passages 34, for example by press-fitting or adhesive bonding. The longitudinal axis of the stiffening pins 35 are closer to the central axis of the bending section body than an inner surface of the circumferential wall 31. FIG. 10 also shows that the passages for the working channel tube and the wires can be merged to form one passage, at least in some segments. Of course, the previously shown transverse wall designs can also be used here.

FIG. 11 shows the cut-out 33 and bend limiting shoulders 51 that are designed, extending from the transverse wall 30 or the circumferential wall 31, to touch and thus limit the bend angle of the passive segment. The stiffening pin 35 is also shown. The stiffening pin 35 can be designed with the desired amount of stiffness, or resistance to bending, by choosing appropriate metal or polymer structures. Coils spring wires can be used, for example. Polymer structures can comprise reinforcing carbon fibers. The bending section sleeve is positioned over the bending section body to seal the bending section body.

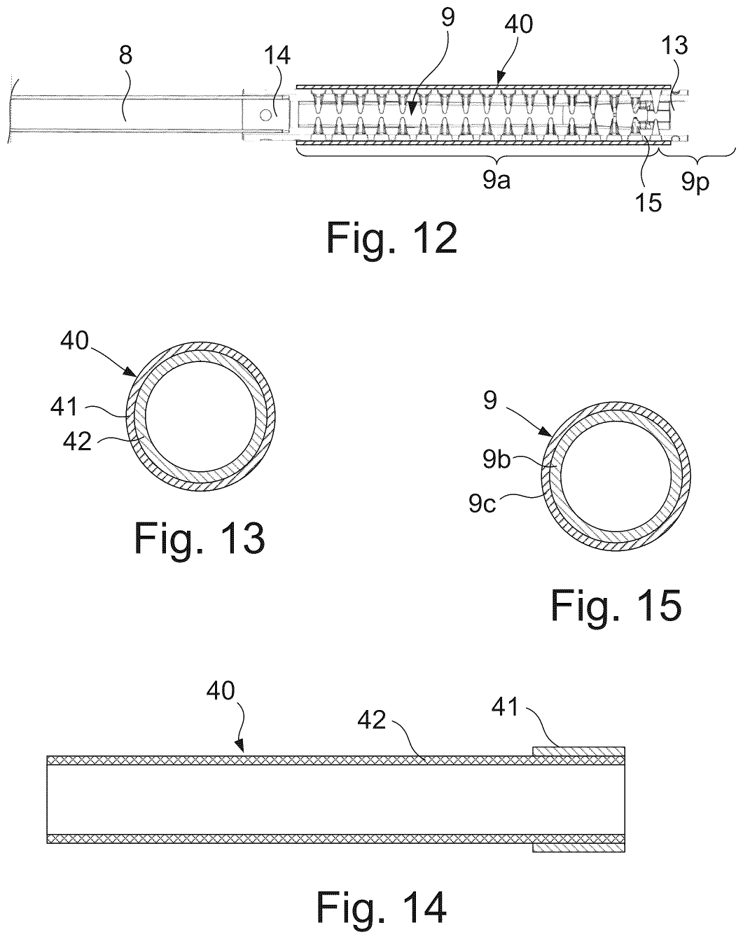

FIG. 12 shows a longitudinal cross-section of the bending section of FIG. 2 and also a longitudinal cross-section of a stiffening bending section sleeve 40. FIGS. 13 and 14 show cross-sections of the stiffening bending section sleeve 40. As shown, a portion of the sleeve overlapping the passive segments is stiffened, thereby the hinges themselves are not necessarily stiffened, but the same effect results from stiffening the sleeve. The sleeve 40 has two layers, 41 and 42, at the stiffening portion and only one layer, 42, overlapping the active segments. Of course, stiffening can also be achieved by maintaining both layers over the entire length of the sleeve but changing the layer thicknesses so that more material is provided over the passive segments. The two layers may be coextruded. Alternatively, one layer may be over-molded over the other layer. Alternatively, one layer may comprise a short sleeve positioned over a longer sleeve. The shorter, stiffening, layer can be inside or outside the longer layer. The result is that a distal portion of the sleeve is stiffer than a proximal portion. In FIG. 12, the passive section 9p of the bending section assembly (body and sleeve) is shown distal of the active section 9a.

FIG. 15 is a cross-sectional view of a segment in the passive section 9p of the bending section body 9, illustrating a two-layer structure having layers 9b and 9c. One of the two layers is a stiffening layer provided inside or outside the layer that includes the active section of the bending section body 9. Construction methods mentioned above with reference to the stiffened sleeve can be used here.

The foregoing means to stiffen the passive hinges can be combined, such as by increasing the width and shortening the length. Additionally, instead of one wider hinge two narrower hinges can be formed side-by-side, and the structure of the pair increases stiffness, in the same manner that a wider hinge is stiffer than a narrower one. An advantage of side-by-side hinges is that it may be easier to control heat dissipation during injection molding.

It should be understood that the advantages of the invention are not only applicable to live hinges but also to any hinges. For example, stiffening pins and stiffened sleeves can be used with traditional bending sections, polymeric or metal, to stiffen hinges connecting segments distal of the steering wires.

The contact force between the ultrasound lens surface and the tissue is defined primarily by the stiffness of the passive links/hinges. Hereby the force on the tissue will be defined by the product and not the user, which can be a benefit. In case the user wants to override the “spring force” on the tissue, there can be made maximum stops on the passive links/hinges so that if the user forces the ultrasound head so much that the passive links are completely closed/collapsed, then the remaining bending force from the user will be transferred to the tissue. The stops can be provided by shaping the cut-outs that form the segments at angles, or with shoulders, that contact each other at a predetermined angle, thus the cut-outs at the passive hinges can be designed for a predetermined overall passive bend angle of the passive section of the bending section.

In a preferred embodiment, the passive hinges are proximal to the exit of the working channel, the camera and the ultrasound transducer. The relative positions of the working channel/tool and the camera and the ultrasound transducer will not be affected by the passive bending. Hereby the tool will always come out in the same position in the ultrasound image, and the tool will always come out in the same position in the camera image and the ultrasound head will always be in the same position relative to the camera image.

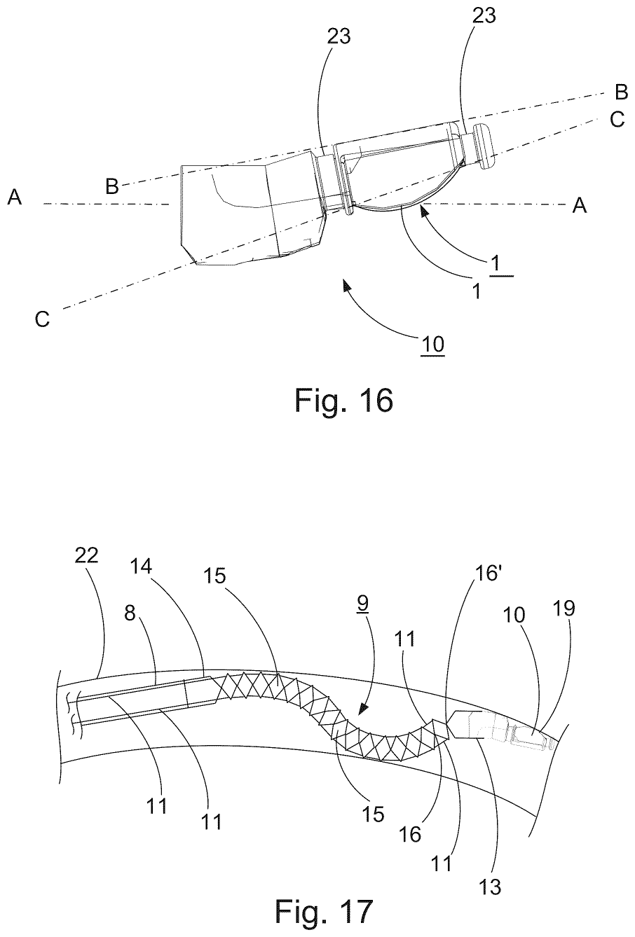

FIG. 16 shows a more detailed side view of the tip part 10. During assembly of the endoscope 1, the tip part 10 is adhered to the distal end segment 13 of the bending section body 9 so that the axis A-A is aligned with the axis A-A shown in FIG. 4. The axis A-A also constitutes the center axis of the generally cylindrical main tube 8 and defines the overall longitudinal axis of the endoscope 1. As can be seen, the tip part 10 is asymmetrical in the sense that the curved emission and reception surface 19′ of the ultrasound transceiver 19 lies more or less on and is at least intersected by the central axis A-A. This provides the ultrasound transceiver 19 with an emission or reception field pointing not only sideways but also somewhat in the forward direction, i.e. with respect to the insertion direction of the endoscope 1, beyond the distal end. This is illustrated by the chord C-C between the ends of the emission and reception surface of the ultrasound transceiver 19, as well as the inclination of the opposite side of the tip part 10, indicated by the axis B-B, with respect to the center axis A-A.

As can be seen, the tip part 10 comprises a pair of recesses 23 adapted to receive ends of a generally tubular balloon (not shown) which may be filled with a suitable liquid, such as water or aqueous saline solution to avoid any air trapped between the ultrasound transceiver 19 that could provide undesired sound reflections due to poor match of acoustic impedance between the ultrasound transceiver 19 and tissue, such as a bronchial wall 22. In other embodiments, there may be only a single recess 23 or groove receiving the balloon.

FIG. 17 is a highly schematic illustration of the engagement of the ultrasound transceiver 19 in the tip part 10 with the bronchial wall 22. It is emphasized that the illustration is schematic and inter alia does not show any deformation of the bronchial wall 22 from the pressure applied thereto by the bending section body 9 and the tip part 10. As can be seen the hinge 16′ between the distal end segment 13 and the last, most distal intermediate segment 15 bends in the opposite direction of the hinge 16 between most distal segment 15 and the penultimate segment 15 before it.

This allows the tip part 10 to obtain a good engagement position with respect to the bronchial wall 22 and with respect to the procedure to be performed, inter alia involving the capture of ultrasound and optical images, as well as the taking of biopsies or the like via the working channel. The rigidity of the hinges 16 arising from the fact that they rely on bending of the material from which the bending section body 9 is made, rather than low friction pivotal hinges as often used in re-usable endoscopes, thus helps securing this good engagement.

Although the above description has been given based on an EBUS endoscope, the skilled person will understand that the concept of one or more passive links/segments in the bending section of an endoscope is not limited to EBUS endoscopes but will be applicable for any endoscope where there may be an interest in a firm aligned sideways abutment of the tip against an object, such as a cavity wall. Such endoscopes may be with or without ultrasound capabilities and could inter alia include EUS endoscopes for gastric purposes or duodenoscopes.

A positioning interface, or interface, functions to control the position of the insertion cord. The handle 6 is an example of a positioning interface and, unless stated otherwise, the terms are used interchangeably. The positioning interface also functions to provide the steering controls, e.g. knobs, levers, buttons, and the like, to steer the field of view of the camera and the elevator controls. Alternatively, a different positioning interface can be provided that is connected to the insertion cord and is detachably connected to a robotic arm. The insertion cord thus extends from the robotic arm, and the intrusive medical device is detachable from the robotic arm. The robotic arm responds to signals, including voice commands from an operator, to rotate, translate, and otherwise position the proximal end of the insertion cord, as an operator would do manually. The positioning interface can include control actuators, including manual control actuators. Alternatively or additionally, control actuators can be provided in or on the robotic arm or by the robotic system including the robotic arm, thereby potentially reducing the cost of the intrusive medical device. Example control actuators include single axis actuators, including linear motion actuators. A linear motion actuator may comprise a threaded rod coupled to a threaded nut portion, in which a motor rotates the rod to translate the nut portion.

The display device 2 may also be referred to as a video processing apparatus (VPA) including a housing enclosing and supporting a display screen, a video processing circuit, and an endoscope interface configured to communicate with the camera at the distal tip. The VPA allows an operator to view an image captured by the image sensor of the camera.

Variations of the VPA can be provided. For example, it might not be desirable to provide a video display screen with a touch screen, or it might be desirable to omit a display screen altogether. Omission of the display screen might be beneficial to take advantage of evolving video display technologies which improve resolution and reduce cost. Provision of exchangeable medical device interfaces allows for adoption of evolving image sensor and endoscope technologies, thus use of existing or future-developed external video displays could allow presentation of higher resolution or otherwise improved video. Use of external video displays could also leverage existing capital investments.

In all embodiments, the endoscope may be disposable and may not be intended to be cleaned and reused. Alternatively, the endoscope may, in all embodiments, be re-usable. In some variations of the present embodiment, the endoscope and the VPA comprise wireless transceivers to exchange image data and configuration data. The endoscope may comprise a battery to power the image sensor and the LEDs.

The video processing circuit of the VPA is operable to receive image data, present a graphical user interface to allow a user to manipulate image data with a touch screen, and, optionally, output a video signal to allow remote viewing of the images presented with the touch screen. A separate, potentially remote, display screen may also be connected to the endoscope via the VPA, which may include or omit the display screen. Medical device interfaces include cable sockets and circuits to compatibilize the signals from the image sensors, for example. Thus, a particular type of endoscope is matched with a corresponding medical device interface and the VPA can thus enable use of different endoscope technologies. In other words, the VPA or monitor is customized to work with endoscope. The medical device interfaces may also include isolation amplifiers to electrically isolate the video signal, and a power output connector to provide power to the endoscope for the image sensor and the LEDs. The medical device interfaces may also include a serial to parallel converter circuit to deserialize the video signals of endoscopes that generate serial signals, for example serial analog video signals. The medical device interfaces may also include a configuration connector to output image sensor configuration parameters such as image inversion, clock, shutter speed etc.

The following items are further variations and examples of the embodiments described with reference to the figures.
1. An endoscope comprising a proximal handle, an insertion cord extending from said handle towards a distal end of the endoscope, where said insertion cord comprises a main tube, a tip part, and a bending section body arranged between said main tube and said tip part, where the bending section body comprises a number of segments interconnected by hinge members arranged in a hinge plane, said segments comprising a proximal end segment, a distal end segment and a number of intermediate segments, where the proximal end segment and at least some of said number of intermediate segments comprise pull-wire passages, wherein said pull-wires are attached and secured to one of said intermediate segments.
2. An endoscope according to item 1, wherein the pull-wire passages arranged in pairs in a plane perpendicular to said hinge plane.
3. An endoscope according to any one of the preceding items, where said number of intermediate segments comprise a first set of intermediate segments through which the pull-wires pass, and a second set of intermediate segments through which the pull-wires do not pass, said second set of intermediate segments being arranged between said first set of intermediate segments and said distal end segments, wherein the pull-wires are connected to the most distal intermediate segment of the first set of segments.
4. An endoscope according to item 3, wherein the number of intermediate segments in said second set of intermediate segments is selected from the group comprising 1, 2, 3, 4 or 5.
5. An endoscope according to any one of the preceding items, wherein the bending section body is provided as an integrally molded item and the hinges are provided as part of the integrally molded item.
6. An endoscope according to any one of the preceding items, wherein the hinge between the distal end segment is thicker than at least some of the hinges between intermediate segments.
7. An endoscope according to any one of the preceding items wherein the tip part comprises an ultrasound transceiver.
8. A system comprising a display unit and an endoscope according to any one of the preceding items connectable to said display unit.
9. A system according to item 8, wherein the display unit comprises an integrated display.
10. A system according to any one of items 8 to 9, further comprising an ultrasound control box for sending signals to an ultrasound transceiver and receiving signals from an ultrasound transceiver.
11. A system according to item 10, wherein the ultrasound control box comprises image processing electronics.

1. An endoscope comprising:
a handle; and
an insertion cord extending distally from the handle, the insertion cord comprising a main tube, a tip part, and a bending section body arranged between the main tube and the tip part, the bending section body comprising segments interconnected by hinges, the segments including a proximal end segment connected to the main tube, a distal end segment connected to the tip part, and intermediate segments between the distal end segment and the proximal end segment; and
a steering wire,
wherein at least one of the intermediate segments comprises a steering wire passage, the steering wire extending from the handle through the main tube and through the steering wire passage,
wherein the steering wire is attached to one of the intermediate segments, and
wherein the steering wire does not extend through segments distal of the one of the intermediate segments to which the steering wire is attached.

2. The endoscope of claim 1, wherein the steering wire passage is arranged in a plane perpendicular to a plane on which the hinges lie.

3. The endoscope of claim 1, where the segments comprise a first set of segments, through which the steering wire passes, and a second set of segments, through which the steering wire does not pass, said second set of segments being arranged between said first set of segments and said distal tip, wherein the steering wire is connected to a most distal segment of the first set of segments.

4. The endoscope of claim 3, wherein the number of segments in said second set of segments is selected from the group comprising 1, 2, 3, 4 or 5.

5. The endoscope of claim 3, wherein the number of segments in said second set of segments is one.

6. The endoscope of claim 1, where the segments comprise a first set of segments, through which the steering wire passes, and at least one segment through which the steering wire does not pass, wherein the steering wire is connected to a most distal segment of the first set of segments.

7. The endoscope of claim 6, wherein the bending section comprises the bending section body and a bending section sleeve positioned over the bending section body, wherein the bending section comprises an active portion and a passive portion distal of the active portion, the first set of segments being in the active portion and the at least one segment through which the steering wire does not pass being in the passive portion, wherein the passive portion of the bending section comprises at least one stiffening feature configured to increase resistance to bending relative to the bending section without the stiffening feature.

8. The endoscope of claim 7, wherein the hinges are live hinges and the stiffening feature comprises live hinges that are stiffer than live hinges in the active portion.

9. The endoscope of claim 7, wherein the stiffening feature comprises stiffening pins positioned coextensivelly with hinges in the passive portion.

10. The endoscope of claim 7, wherein the stiffening feature comprises a stiffening layer of material in the passive portion, in the bending section body and/or the bending section sleeve.

11. The endoscope of claim 6, wherein the first set of segments form an active portion of the bending section and the at least one segment through which the steering wire does not pass forms a passive portion of the bending section, wherein the hinges are live hinges and the live hinges in the passive portion of the bending section are more resistant to bending than the live hinges in the active portion of the bending section.

12. The endoscope of claim 6, wherein the first set of segments form an active portion of the bending section and the at least one segment through which the steering wire does not pass forms a passive portion of the bending section, wherein the passive portion of the bending section comprises stiffening pins extending coextensivelly with the hinges, the stiffening pins increasing a resistance to bending.

13. The endoscope of claim 12, wherein the hinges are live hinges.

14. The endoscope of claim 1, wherein the hinges are live hinges, wherein live hinges between the distal end segment and a most distal intermediate segment are thicker than at least some of the live hinges between intermediate segments proximal of the most distal intermediate segment.

15. The endoscope of claim 1, wherein the tip part comprises an ultrasound transceiver.

16. A system comprising a display unit and the endoscope of claim 1, the endoscope being connectable to said display unit.

17. The system of claim 16, wherein the display unit comprises an integrated display.

18. The system of claim 16, wherein the tip part comprises an ultrasound transceiver, the system further comprising an ultrasound control box for sending signals to an ultrasound transceiver and receiving signals from an ultrasound transceiver.

19. The system of claim 16, wherein the ultrasound control box comprises image processing electronics.
